(19) United States
(12) Patent Application Publication
(10) Pub. No.: US 2010/0234604 A1
(43) Pub. Date: Sep. 16, 2010

(54) Process for Making Substituted Aryl Sulfone Intermediates

(76) Inventors: Xin Linghu, Edison, NJ (US); Kathleen Linn, North Brunswick, NJ (US); Kevin M. Maloney, Piscataway, NJ (US); Mark McLaughlin, Summit, NJ (US); Gang Qian, Kendall Park, NJ (US)

Correspondence Address: MERCK, P O BOX 2000, RAHWAY, NJ, 07065-0907, US

(21) Appl. No.: 12/722,711

(22) Filed: Mar. 12, 2010

Related U.S. Application Data
(60) Provisional application No. 61/210,087, filed on Mar. 13, 2009.

(51) Int. Cl. C07D 211/20; C07D 213/62

(52) U.S. Cl. 546/248; 546/294

(57) ABSTRACT

The present invention relates to novel substituted aryl sulfone intermediates and processes for preparing the same. An aspect of this invention relates to a process for making substituted aryl sulfone intermediates utilizing a one-pot deacylation-carbon/sulfur bond formation step. The invention also relates to a process for preparing intermediates that are used to make the compounds of formula I. Some of the advantages of the present invention include manufacturing flexibility and efficiency, high yield synthesis using a one pot deacylation and carbon-sulfur bond formation step of a thioester intermediate and the like. This and other aspects of the invention will be realized upon review of the specification as a whole.

### BACKGROUND TO THE INVENTION

The invention disclosed herein concerns substituted aryl sulfones, intermediates and process for synthesis thereof. In particular, this invention relates to novel substituted aryl sulfone intermediates and processes for making the intermediates. Substituted aryl sulfone compounds are known to be N-type calcium channel (Cav2.2) blockers useful for the treatment of acute pain, chronic pain, cancer pain, visceral pain, inflammatory pain, neuropathic pain, post-herpetic neuralgia, diabatic neuropathy, trigeminal neuralgia, migrane, fibromyalgia and stroke. The compounds also are known to display activities on T-type voltage-activated calcium channels (Cav 3.1 and Cav 3.2). See for example U.S. Ser. No. 60/997,615 (Attorney Docket #22454PV), U.S. Pat. Nos. 6,011,035; 6,294,533; and 6,617,322; and publication numbers WO2007/075525, US2004/044004, JP2002/088073, WO2007085357, W2007028638, WO94/22835, US20030408, and WO2004/096217, WO2004/031138, WO2003084948, WO2003/075853, WO2001/025200, WO2007056075, WO2005000798 and WO2002/055516.

### SUMMARY OF THE INVENTION

The present invention relates to novel substituted aryl sulfone intermediates and processes for preparing the same. An aspect of this invention relates to a process for making substituted aryl sulfone intermediates utilizing a one-pot deacylation-carbon/sulfur bond formation step. The invention also relates to a process for preparing additional intermediates used to make the compounds of formula I below. Some of the advantages of the present invention include manufacturing flexibility and efficiency, high yield synthesis using a one pot deacylation and carbon-sulfur bond formation step of a thioester intermediate and the like. This and other aspects of the invention will be realized upon review of the specification as a whole.

### DETAILED DESCRIPTION OF THE INVENTION

The instant invention relates to a process for making sulfonamide compounds of formula I, which are described in PCT/US08/11286:

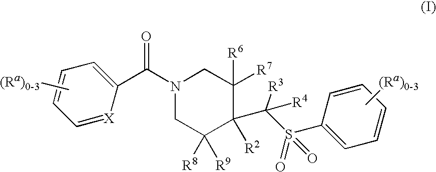

and pharmaceutically acceptable salts thereof and individual enantiomers and diastereomers thereof, wherein:

### X is CH or N;

R¹ is H, C₁₋₆-alkyl, C₃₋₇-cycloalkyl, OR¹⁰, C(O)R¹⁰, (CH₂)ₙC₅₋₁₀ heterocycle, (CH₂)ₙC₆₋₁₀ aryl, (CH₂)ₙC₅₋₁₀ heteroaryl, fused aryl or fused heteroaryl, wherein said alkyl, cycloalkyl, heterocycle, aryl and heteroaryl is optionally substituted with one to three groups of R^{a}; R² is H, C₁₋₄ alkyl and C₁₋₄-perfluoroalkyl, C³⁻⁵-cycloalkyl, C₆₋₁₀ aryl, C₅₋₁₀ heteroaryl, F, Cl, CN, NR¹⁰R¹¹, wherein said alkyl, cycloalkyl, aryl and heteroaryl is optionally substituted with one to three groups of R^{a};
R³ and R⁴ are each and independently selected from H, or C₁₋₆ alkyl, C₁₋₄-perfluoroalkyl, C₃₋₇-cycloalkyl, C₆₋₁₀ aryl, C₅₋₁₀ heteroaryl, F, Cl, CN, OR¹⁰, NR¹⁰R¹¹, SO₂R¹⁰, SO₂NR¹⁰R¹¹, CO₂R¹⁰, CONHR¹⁰, CONR¹⁰R¹¹, or R³ and R⁴ join to form a 3-7 member carbocyclic or heterocyclic ring, wherein said alkyl, cycloalkyl, heterocycle, aryl and heteroaryl is optionally substituted with one to three groups of R^{a};
R⁵ is C₆₋₁₀ aryl, C₅₋₁₀ heteroaryl, C₃₋₇ cycloalkyl, C₅₋₁₀ heterocycle, wherein said cycloalkyl, heterocycle, aryl and heteroaryl is optionally substituted with one to three groups of R^{a};
R⁶, R⁷, R⁸, and R⁹ independently represent H, C₁₋₄alkyl and C₁₋₄ perfluoroalkyl, C₃₋₆-cycloalkyl, C₆₋₁₀ aryl, C₅₋₁₀ heteroaryl, F, Cl, CN, OR¹⁰, NR¹⁰R¹¹, or R⁸ and R⁹ combined with the carbon atom they are attached to can form C(O);

R¹⁰ and R¹¹ are each and independently selected from H, or C₁₋₆alkyl, (CH₂)ₙC₁₋₄-fluoroalkyl, C₃₋₇cycloalkyl, C₆₋₁₀ aryl, C₅₋₁₀ heteroaryl, or R¹⁰ and R¹¹ join to form a 3-7 member carbocyclic or heterocyclic ring with the atom to which they are attached; said alkyl, aryl, or heteroaryl optionally substituted with 1 to 3 groups of R^{a},

n represents 0 to 6, and
R^{a} represents C₁₋₆ alkyl, C₃₋₇ cycloalkyl, C₁₋₄-fluoroalkyl, C₆₋₁₀ aryl, C₅₋₁₀ heteroaryl, halogen, CN, —OCF₃, —OCHF₂, —C(O)CF₃, —C(OR¹⁰)(CF₃)₂, SR¹⁰, —OR¹⁰, NR¹⁰R¹¹, SOR¹⁰, SO₂R¹⁰, NR¹⁰COR¹¹, NR¹⁰COOR¹¹, NR¹⁰CONR¹⁰R¹¹, NR¹⁰SO₂NR¹⁰R¹¹, SO₂NR¹⁰R¹¹, NR¹⁰SO₂R¹¹, CO₂R¹⁰, CONR¹⁰R¹¹, said aryl and heteroaryl optionally substituted with 1 to 3 groups of C₁₋₆ alkyl, C₃₋₇ cycloalkyl, halogen, CF₃, CN or OR¹⁰;
comprising the steps of:
(1) one pot deacylation of a compound of formula 3, and formation of a carbon-sulfur bond with a compound of formula 4

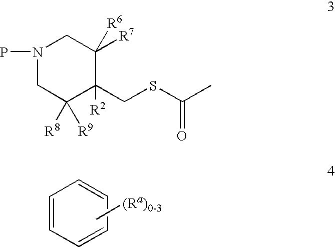

in the presence of a first base, first metal catalyst and ligand to produce a compound of formula 5, wherein P is an amino protecting group:

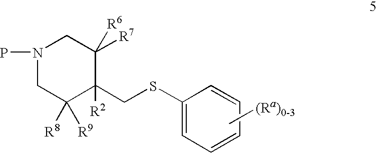

(2) oxidation of the compound of formula 5 using an oxidizing agent to produce a compound of formula 6:

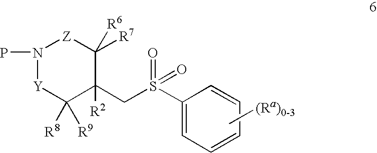

(3) alkylation of the compound of formula 6 using a second base at a temperature of about −20° C. to about −100° C. to produce a compound of formula 7:

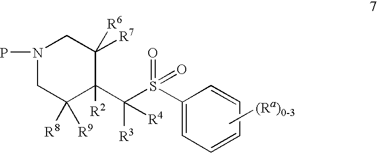

(4) deprotection of the compound of formula 7, purification and isolation of the compound of formula 8:

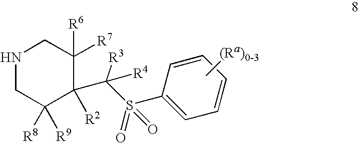

(5) coupling a compound of formula 8 with a compound of formula II:

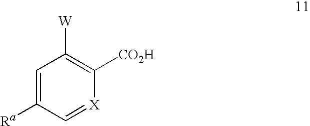

and pharmaceutically acceptable salts, individual enantiomers and diastereomers thereof wherein R^{a} is previously described, W is selected from the group consisting of C₁₋₆ alkyl, C₃₋₇ cycloalkyl, C₁₋₄-fluoroalkyl, halogen, CN, —OCF₃, SR¹⁰, —OR¹⁰, NR¹⁰R¹¹, SOR¹⁰, SO₂R¹⁰, NR¹⁰COR¹¹, NR¹⁰COOR¹¹, SO₂NR¹⁰R¹¹, NR¹⁰SO₂R¹¹, CO₂R¹⁰, and CONR¹⁰R¹¹, and X is CH or N, in the presence of a third base, and peptide forming reagent, purifying and isolating to produce a compound of formula I.

The compounds of formula I are substituted aryl sulfone derivatives that are N-type voltage-gated calcium channel blockers useful for the treatment of a variety of pain conditions including acute and chronic pain such as neuropathic, inflammatory, and visceral pain. The compounds also display activity in connection with blockage of T-type voltage-gated calcium channels.

When any variable (e.g. aryl, heterocycle, R¹, R⁵ etc.) occurs more than one time in any constituent, its definition on each occurrence is independent at every other occurrence. Also, combinations of substituents/or variables are permissible only if such combinations result in stable compounds.

When R^{a} is —O— and attached to a carbon it is referred to as a carbonyl group and when it is attached to a nitrogen (e.g., nitrogen atom on a pyridyl group) or sulfur atom it is referred to a N-oxide and sulfoxide group, respectively.

As used herein, “alkyl” encompasses groups having the prefix “alk” such as, for example, alkoxy, alkanoyl, alkenyl, and alkynyl and means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl. Preferably, alkenyl is C₂-C₆ alkenyl. Preferred alkynyla are C₂-C₆ alkynyl.

“Alkenyl,” “alkynyl” and other like terms include carbon chains containing at least one unsaturated C—C bond.

As used herein, “fluoroalkyl” refers to an alkyl substituent as described herein containing at least one fluorine substituent.

The term “cycloalkyl” refers to a saturated hydrocarbon containing one ring having a specified number of carbon atoms. Examples of cycloalkyl include cyclopropyl, cyclobutyl, cyclopentyl, and cyclohexyl.

The term “C₁₋₆” includes alkyls containing 6, 5, 4, 3, 2, or 1 carbon atoms

The term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxyethane (also referred to as ‘ethyl methyl ether’).

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl. The term heterocycle, heterocyclyl, or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocycle or heterocyclic includes heteroaryl and heterocycloalkyl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

In certain embodiments, the heterocyclic group is a heteroaryl group. As used herein, the term “heteroaryl” refers to groups having 5 to 14 ring atoms, preferably 5, 6, 9, or ring atoms; having 6, 10, or 14 π electrons shared in a cyclic array; and having, in addition to carbon atoms, between one and about three heteroatoms selected from the group consisting of N, O, and S. heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

In certain other embodiments, the heterocyclic group is fused to an aryl or heteroaryl group. Examples of such fused heterocycles include, without limitation, tetrahydroquinolinyl and dihydrobenzofuranyl.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

Examples of heterocycloalkyls include azetidinyl, pyrrolidinyl, piperidinyl, piperazinyl, morpholinyl, tetrahydrofuranyl, imidazolinyl, pyrrolidin-2-one, piperidin-2-one, and thiomorpholinyl.

The term “heteroatom” means O, S or N, selected on an independent basis. A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2,4-fluor-3-propylphenyl. As another non-limiting example, substituted n-octyls include 2,4 dimethyl-5-ethyl-octyl and 3-cyclopentyloctyl. Included within this definition are methylenes (—CH₂—) substituted with oxygen to form carbonyl (—CO—).

Unless otherwise stated, as employed herein, when a moiety (e.g., cycloalkyl, hydrocarbyl, aryl, alkyl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—), nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino, and
(b) C₁-C₆ alkyl or alkenyl or arylalkyl imino, carbamoyl, azido, carboxamido, mercapto, hydroxy, hydroxyalkyl, alkylaryl, arylalkyl, C₁-C₈ alkyl, SO₂CF₃, CF₃, SO₂Me, C₁-C₈ alkenyl, C₁-C₈ alkoxy, C₁-C₈ alkoxycarbonyl, aryloxycarbonyl, C₂-C₈ acyl, C₂-C₉ acylamino, C₁-C₈ alkylthio, arylalkylthio, arylthio, C₁-C₈alkylsulfinyl, arylalkylsulfnyl, arylsulfnyl, C₁-C₈ alkylsulfonyl, arylalkylsulfonyl, arylsulfonyl, C₀-C₆ N-alkylcarbamoyl, C₂-C₁₅ N,N dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, or aryl, wherein each of the foregoing is further optionally substituted with one more moieties listed in (a), above.

“Halogen” refers to fluorine, chlorine, bromine and iodine.

The term “mammal” “mammalian” or “mammals” includes humans, as well as animals, such as dogs, cats, horses, pigs and cattle.

Compounds described herein may contain one or more double bonds and may thus give rise to cis/trans isomers as well as other conformational isomers. The present invention includes all such possible isomers as well as mixtures of such isomers unless specifically stated otherwise.

The compounds disclosed in the present invention may contain one or more substituents, and asymmetric centers and may thus occur as racemates, racemic mixtures, single enantiomers, diastereomeric mixtures, and individual diastereomers.

It will be understood that, as used herein, references to the compounds of structural formula I are meant to also include the pharmaceutically acceptable salts, and also salts that are not pharmaceutically acceptable when they are used as precursors to the free compounds or in other synthetic manipulations.

The individual processes within the general process can be summarized in Scheme I as follows:

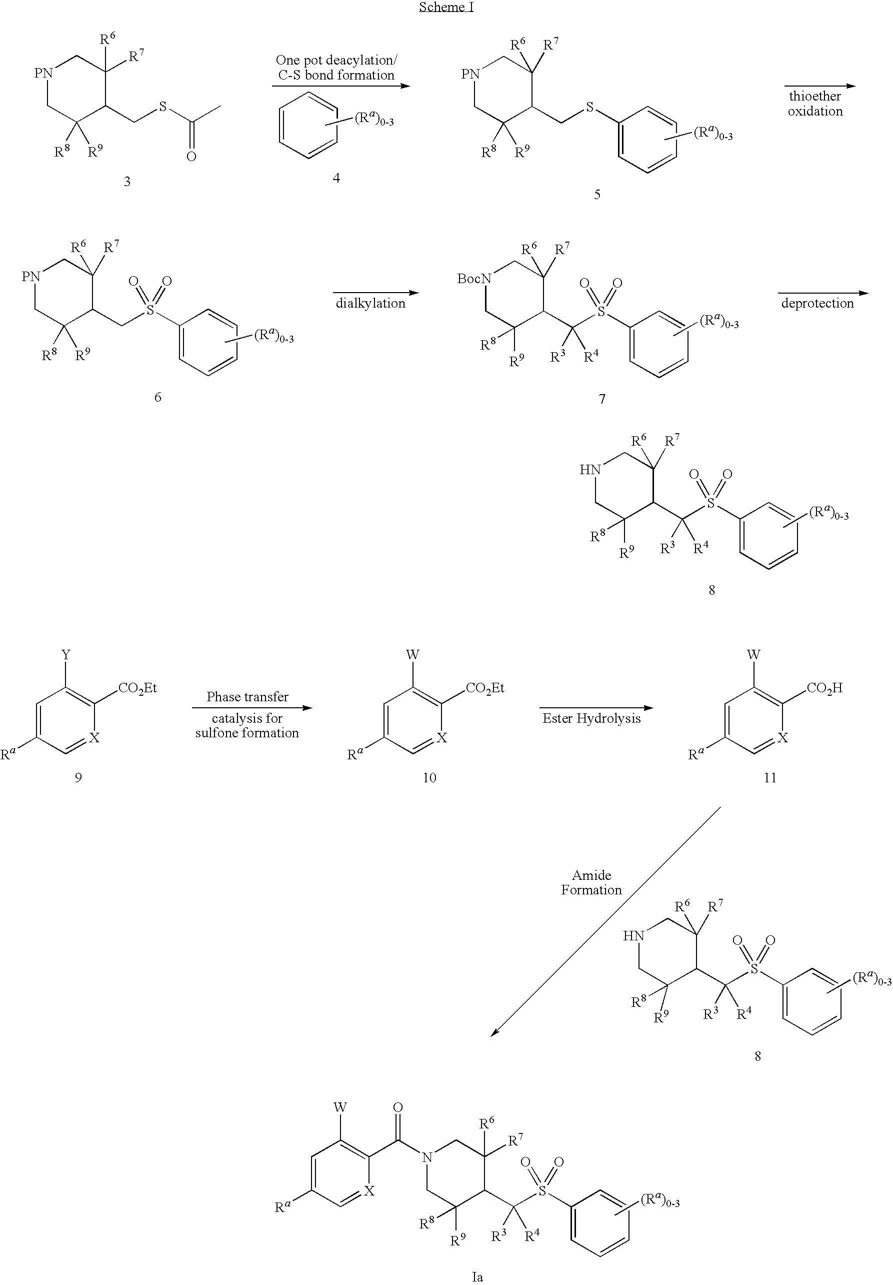

wherein P is an appropriate amino protecting group, W is C₁₋₆ alkyl, C₃₋₇ cycloalkyl, C₁₋₄-fluoroalkyl, halogen, CN, —OCF₃, SR¹⁰, —OR¹⁰, NR¹⁰R¹¹, SOR¹⁰, SO₂R¹⁰, NR¹⁰COR¹¹, NR¹⁰COOR¹¹, SO₂NR¹⁰R¹¹, NR¹⁰SO₂R¹¹, CO₂R¹⁰, or CONR¹⁰R¹¹, Y is Cl, Br, F, I or OTf, X is CH, or N, and R^{a}, R³, R⁴, R⁶, R⁷, R⁸, R⁹, R¹⁰, and R¹¹ are as previously described.

Within this general process, an embodiment of this process concerns the preparation of a compound of formula 8:

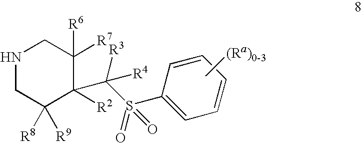

and pharmaceutically acceptable salts, individual enantiomers and diastereomers thereof wherein R^{a}, R², R³, R⁴, R⁶, R⁷, R⁸, and R⁹ are previously described, comprising the steps of:
(1) one pot deacylation of a compound of formula 3, and formation of a carbon-sulfur bond with a compound of formula 4

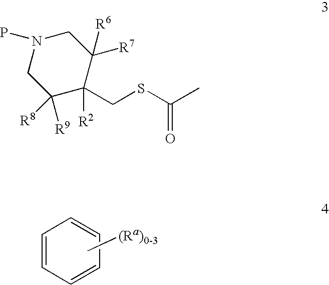

in the presence of a first base, first metal catalyst and ligand to produce a compound of formula 5, wherein P is an amino protecting group:

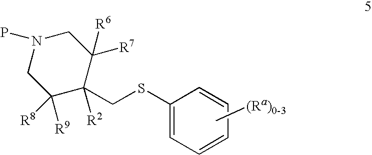

(2) oxidation of the compound of formula 5 using an oxidizing agent to produce a compound of formula 6:

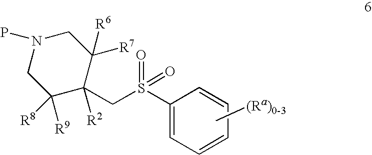

(3) dialkylation of the compound of formula 6 using a second base at a temperature of about −20° C. to about −100° C. to produce a compound of formula 7:

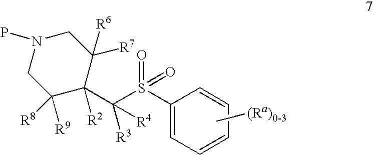

(4) deprotection of the compound of formula 7, purification and isolation of the compound of formula 8.

Appropriate solvents for this process include those which will at least partially dissolve one or all of the reactants and will not adversely interact with either the reactants or the product. Suitable solvents are aromatic hydrocarbons (e.g, toluene, xylenes), halogenated solvents (e.g, methylene chloride, chloroform, carbontetrachloride, chlorobenzenes), ethers (e.g, diethyl ether, diisopropylether, tert-butyl methyl ether, diglyme, tetrahydrofuran, dioxane, anisole), nitriles (e.g, acetonitrile, propionitrile), ketones (e.g, 2-butanone, dithyl ketone, tert-butyl methyl ketone), alcohols (e.g, methanol, ethanol, n-propanol, iso-propanol, n-butanol, t-butanol), N,N-dimethyl formamide (DMF), dimethylsulfoxide (DMSO) and water. Mixtures of two or more solvents can also be used.

Suitable first bases for this process include: alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, potassium carbonate, sodium carbonate, cesium carbonate, sodium hydrogen carbonate, and cesium hydrogen carbonate; phosphates such as calcium phosphate, sodium phosphate, and the like.

Suitable first metal catalysts are those which contain a metal known to be useful for catalytic hydrogenation such as palladium (Pd). The metal catalyst can be a salt or metal powder or supported on a wide range of solid supports known to be useful in catalytic hydrogenation reactions including alumina, silica, calcium carbonate, barium carbonate, barium sulfate, strontium carbonate, polymers, or carbon, preferably activated carbon. The catalyst is used in an amount that is at least 0.2-5 mol % relative to the substrate, preferably 1%. Metal catalyst suitable for this process include, but are not limited to, palladium (II) acetate, tetrakis(triphenylphospine)palladium (O), tris(dibenzylideneacetone) dipalladium, tetradibenzylideneacetone)dipalladium, Palladium on carbon, palladium (II) halide and the like. See, for example, Beller et al. Angew. Chem. Int. Ed. Engl., 1995, 34 (17), 1848.

Suitable ligands for the first metal catalyst in this process include, but are not limited to, chiral monodentate or polydentate, which optionally can possess an alkylated or arylated phosphine. Examples of ligands are TetraMe-BITIOP-(TMBTP-see Benincori, T.; Cesarotti, E.; Piccolo, O.; Sannicolo, F. J. Org. Chem., 2000, 65, 2043-2047 for full name); (S)-Me-f-Ketalphos-((3aS,3′ aS,4S,4′S,6S,6′S,6aS,6′aS)-5,5′-[1,1′-feaocenyl]bis[tetrahydro-2,2,4,6-tetramethyl-4H-phospholo[3,4-d]-1,3-dioxole] see Liu, D.; Li, W.; Zhang, X. Organic Letters, 2002, 4, 4471-4474); (S)-BINAP; (R,R)-Et-ferrotane; (R)-xylBINAP; (R)-phanephos; (S)-Binaphane; (R)-xylPhanephos; (R,S)-Tangphos; (S)-Me-BoPhoz; (5,5)-Norphos; (R,R)-Me-DuPhos; (R,S)-((diphenylphosphino)ferrocenyl-ethyldicyclohexylphosphine); tBu-Josiphos-((R,S)-((diphenylphosphino)ferrocenyl-ethyldi-t-butylphosphine) see Togni, A.; Breutel, C.; Schnyder, A.; Spindler, F.; Landert, H.; Tijani, A. J. Am. Chem. Soc., 1994, 116, 4062-4066); [1,1′-bis(diphenylphosphino)ferrocene] (dppf); (R,S)-((di-t-butylphosphino)ferrocenyl-ethyldi-3,5-dimethylphenylphosphine); triphenylphosphine, bis(diphenylphosphino)methane, bis(diphenylphosphino)ethane, bis(diphenylphosphino)propane, 1,4-bis(diphenylphosphino)butane, bis(diphenylphosphino)pentane, tri-o-tolyl-phosphine, and the like. Preferred ligands are phosphine ligands such as [1,1′-bis(diphenylphosphino)ferrocene] (dppf), (R,S)-((di-t-butylphosphino)ferrocenyl-ethyldi-3,5-dimethylphenylphosphine); (R,S)-((diphenylphosphino)ferrocenyl-ethyldicyclohexylphosphine); and tBu-Josiphos-((R,S)-((diphenylphosphino)ferrocenyl-ethyldi-t-butylphosphine). Examples of metal catalyst-ligand complex include Pd₂(dba)₃/dppf, PdCl₂/Et₃N and the like. The catalyst can be prepared by contacting a transition metal salt or its complex and a ligand via methods known in the art. The catalyst may be prepared in situ or as an isolated compound. The amount of ligand used can be from about 1:1 to about 2:1 that of the catalyst, eg., 2-10 mol % relative to the substrate, preferably 2%.

Step 1 is suitably conducted in an appropriate solvent such as those listed above at a temperature in a range from about 25° C. to about 150° C., typically about 50° C. to about 125° C. and more typically about 50° C. to about 90° C.

Suitable oxidizing agents for this process include, but are not limited to, hydrogen peroxide, acetone, N-bromosaccharin, N-bromosuccinimide, N-tert-butylbenzenesulfinimidoyl chloride, tert-butyl hydroperoxide, tert-butyl hypochlorite, 3-chloroperoxybenzoic acid, cerium ammonium nitrate, hydrogen dimethyl sulfoxide, meta-chloroperbenzoic acid, osmium tetroxide, sodium hyperchlorite, oxone, and the like, preferably meta-chloroperbenzoic acid, and 3-chloroperoxybenzoic acid.

The oxidizing agent in Step 2 can be employed in at least 0.5 to about 10.0 equivalent per mole equivalent of Compound 5. The amount of oxidizing agent is typically in the range of about 1 to about 5 mole equivalents per equivalent of Compound 5. In one embodiment, the amount of oxidizing agent is from about 2.0 to about 2.5 (e.g., 2.0 to about 2.5) mole equivalents per mole equivalent of compound 5.

Step 2 is suitably conducted at a temperature in a range from about −10° C. to about 25° C., typically about −5° C. to about 5° C. and more typically about −2° C. to about 2° C.

Alkylating agents for Step 3 of this process include, but are not limited to, those wherein R³ and R⁴ are selected from methyl, tert-butyl, isopropyl or isobutyl, preferably methyl.

Suitable second bases for this process include: alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO, metal amides, and the like, preferably alkali metal alkoxides and alkaline earth metal alkoxides sodium methoxide, sodium ethoxide, potassium tert-butoxide and magnesium ethoxide.

Step 3 is suitably conducted at a temperature in a range from about −20° C. to about −100° C., typically about −50° C. to about −90° C. and more typically about −60° C. to about −78° C.

The term “amino protecting group,” as used herein, refers to a labile chemical moiety which is known in the art to protect an amino group against undesired reactions during synthetic procedures. After said synthetic procedure(s) the amino protecting group as described herein may be selectively removed Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The amino group can be protected by reaction with a suitable amine protecting reagent in a suitable solvent. Suitable amine protecting reagents for this process include, but are not limited to, (C₁₋₆ alkyloxy)carbonyl halides (Boc halides), di-tert-butyl carbonate, di-allyl carbonate, dibenzyl carbonate, carbobenzyloxy (CBZ), or p-nitrobenzyl carbamoyl (PNZ), benzyloxycarbonyl halides (CBZ halides), allyloxycarbonyl halides (ALLOC halides), diphenylphosphinyl halides, di-(C₁₋₃ alkyl)phosphono halides, diphenylphosphono halides, and dibenzylphosphono halides. Representative examples of amine protecting agents in this class are Ph₂P(═O)Cl, (i-PrO)₂P(═O)Cl, (t-BuO)₂P(═O)Cl, (BnO)₂P(═O)Cl, BOC—Cl, CBZ—Cl, (CBZ)₂O, (ALLOC)₂O, allyl chloroformate, and (BOC)₂O. Particularly suitable amine protecting agents are selected from BOC-halide and (BOC)₂O.

In Step 4 removal of the amino protecting group, such as benzyloxycarbonyl (carbobenzyloxy), group may be achieved by a number of methods, for example, hydrogenolysys, catalytic hydrogenation with hydrogen in the presence of a nobel metal or its oxide such as palladium on activated carbon in a protic solvent such as ethanol. In cases where catalytic hydrogenation is contraindicated by the presence of other potentially reactive functionality, the removal of a benzyloxycarbonyl (carbobenzyloxy) or t-butoxycarbonyl group may also be achieved, for example, by treatment with a solution of hydrogen bromide in acetic acid, treatment with methane sulfonic acid, or treatment with TsOH, or by treatment with a mixture of TFA and dimethylsulfide. Removal of benzyloxycarbonyl protecting group may be carried out in a solvent such as methanol, ethanol, methylene chloride, toluene, ethyl acetate, or iso-propyl actate, with a strong acid. Such strong acids include methanesulfonic acid, trifluoroacetic acid, hydrochloric acid, hydrogen chloride, metal hydroxides, hydrogen bromide, hydrogen idodide, trifluoromethane-sulfonic acid, camphorsulfonic acid, sulfuric acid, phosphoric acid, and arylsulfonic acids, such as benzenesulfonic acid, p-toluenesulfonic acid, and p-chlorobenzene-sulfonic acid. Step 4 is suitably conducted at a temperature of about 20° C. to about 100° C.

With Step 5 coupling (amide formation) can occur utilizing suitable third bases which include: tertiary amine bases (e.g., triethylamine, trimethylamine, aniline, dimethylethanolamine, N-ethyldiisoproylamine (Hunig's Base)), alkali metal carbonates and alkaline earth metal carbonates such as lithium carbonate, potassium carbonate, sodium carbonate, cesium carbonate, sodium hydrogen carbonate, and cesium hydrogen carbonate, 4-methyl morpholine, 4-dialkylamino pyridines and the like, preferably the tertiary amine bases such as Hunig's Base or potassium carbonate, depending on the reaction conditions.

Suitable peptide forming reagents for Step 5 of this process include carbodiimides, pyridium salts, phosphonium salts and uranium salts such as BOP (1H-benzotriazol-1-yloxy-tris(dimethylamino)phosphonium hexafluorophosphate), PyBOP (benzotriazol-1-yl-oxy-tris-pyrrolidinophosphonium hexafluorophosphate), HBTU (2-(1H-benzo-triazole-1-yl)-1,1,3,3,-tetramethyluronium hexafluorophosphate), HATU (2-(1H-7 azabenzotriazole-1-yl)-1,1,3,3,-tetramethyl uronium hexafluorophosphate), TBTU ((2-(1H-benzotriazole-1-yl)-1,1,3,3,-tetramethyluronium tetrafluoroborate), DCC (dicyclohexyl carbodiimide), and EDCl (1-Ethyl-3-(3-dimethyllaminopropyl)carbodiimide hydrochloride), preferably the phosphonium salts such as BOP and PyBOP.

Alternatively, coupling can occur first by the formation of an acyl chloride followed by amide bond formation. This generally is accomplished by reacting compound II with a chlorination reagent in the presence of a solvent to produce the resulting acyl chloride followed by contact with a third base as indicated above. A suitable temperature for this reaction is about 0° C. to about 25° C. Suitable chlorination reagents include oxalyl chloride, SOCl₂ POCl₃, LiCl, BCl₃, AlCl₃, HgCl₂, TiCl₄, t-butyl hypochorite, benzoyl chloride, butyl chloroformate, tosyl chloride and the like.

Suitable solvents for coupling Step 5 of this process include acetonitrile, dichloromethane, dimethylacetamide, dimethylformamide, dimethyl sulfoxide, tetrahydrofuran, trifluoroethanol, 1-methyl-2-pyrollidinone, 1,1,3,3,3-hexafluoro-2-propanol, and the like or mixtures thereof. Step 5 is suitably conducted at a temperature in a range from about −10° C. to about 50° C., typically about −0° C. to about −40° C. and more typically at about room temperature.

Within this general process, a second embodiment of this process concerns the preparation of a compound of formula II:

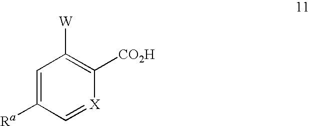

and pharmaceutically acceptable salts, individual enantiomers and diastereomers thereof wherein R^{a}, W and X is previously described, comprising the steps of:
(1a) nucleophilic displacement of the Y substituent in a compound of formula 9:

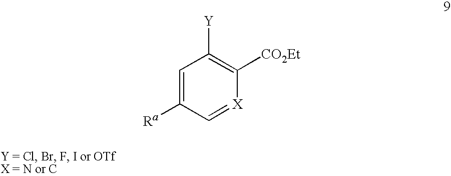

using a nucleophile in the presence of a second catalyst to produce a compound of formula 10, wherein W is previously defined:

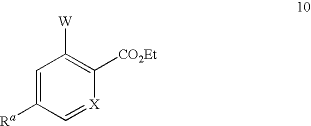

(2a) hydrolysis of the compound of formula 10 in the presence of a fourth base, to produce a compound of formula II, and
(3a) purifying and isolating the compound of formula 11.

In Step 1a the nucleophilic agent can be any agent capable of adding a nucleophilic group to Compound 9 under the reaction conditions employed in Step 1a. Suitable nucleophiles include, but are not limited to, those selected from the group consisting of alkali metal salts of C₁-C₆ alkylsulfonic acids, C₁-C₆ alkylcarboxylic acids, alkaline earth metal salts of C₁-C₆ alkylcarboxylic acids, C₁-C₆ thioalcohols, C₁-C₆ alkylamines, N—(C₁-C₄ alkyl)-C₁-C₆ alkylamines, C₅-C₇ cycloalkylamines, C₅-C₇ azacycloalkanes, alkali metal C₁-C₆ alkoxides, alkali metal amides, and alkali metal cyanides. Exemplary nucleophiles include NaOAc, KOAc, Mg(OAc)₂, NaSO₂Me, sodium proprionate, methanethiol, ethanethiol, methylamine, ethylamine, n-propylamine, cyclopentylamine, piperidine, piperazine, NaOEt, NaOPr, NaNH₂, KNH₂, NaCN, and KCN. In one embodiment, the nucleophile is an alkali metal salt of a C₁-C₆ alkylsulfonic acid (e.g., NaSO₂Me).

The nucleophilic agent can be employed in Step 1a in any proportion with respect to Compound 9 which will result in at least some cleavage of the chloro substituent in compound 9. For example, the amount of nucleophilic agent employed in Step 1a can be at least about 0.5 equivalent per mole equivalent of Compound 9. The amount of nucleophile is typically in the range of from about 0.7 to about 20 mole equivalents per equivalent of Compound 9, and is more typically in the range of from about 1 to about 20 mole equivalents per equivalent of Compound 9. In one embodiment, the amount of nucleophile is from about 1 to about 10 (e.g., from about 1.05 to about 5) mole equivalents per mole equivalent of Compound 9. In another embodiment, the amount of nucleophilic agent is in the range of from about 1.1 to about 4 (e.g., from about 1.1 to about 2) mole equivalents per mole equivalent of Compound 9.

Suitable second catalysts in Step 1a include, but are not limited to, tetrabutyl ammonium chloride, tetrabutyl ammonium bromide, tetrabutyl ammonium bisulphate, tetrapropyl ammonium bromide, tetraethyl ammonium bromide, tetramethyl ammonium bromide, tetrabutylmethyl ammonium chloride, benzyltriethyl ammonium chloride, tricaprylmethyl ammonium chloride, triethyl ammonium methylene bromide, methyltrioctyl ammonium chloride, and the like, preferably, tetrabutyl ammonium chloride, and tetrabutylmethyl ammonium chloride. In general, the second catalyst amount used varies in the range between 0.01% and 50% by weight, preferably, 1% to about 15%, with respect to the reagent in the least polar phase.

Suitable solvents in Step 1a include, but are not limited to, carboxylic acids, amides and esters of carboxylic acids, aliphatic and cyclic ethers and diethers, nitriles, amines, and sulfoxides. Exemplary solvents include polar organic solvent, such as N-ethylpyrrolidinone (NEP), N-methylpyrrolidinone, N,N-dimethylformamide (DMF), N,N-dimethylacetamide (DMA), acetonitrile, propionitrile, or a mixture thereof and the like. Other useful solvents are acetic acid, propionic acid, butyric acid, valeric acid, ethyl ether, methy t-butyl ether, propyl ether, THF, dioxane, acetonitrile, propionitrile, valeronitrile, NMP, DMPU and dimethylsulfoxide. In one embodiment, the solvent is selected from the group consisting of acetic acid, DMF, DMA, and NMP.

Step 1a is suitably conducted at a temperature in a range of from about 20 to about 200° C. (e.g., from about 40 to about 200° C.), and is typically conducted at a temperature in a range of from about 50 to about 130° C. In one embodiment, the temperature is in a range of from about 70 to about 120° C. In another embodiment, the temperature is in the range of from about 90 to about 120° C. (e.g., from about 90 to about 100° C.). The reactants can be added to the reaction vessel (also referred to herein as the reaction “pot”) in Step 1a concurrently, either together or separately, or they can be added sequentially in either order. The solvent can be added before, during, or after addition of Compound 9 or the nucleophile or both Compound 9 and the nucleophile. In one embodiment, Compound 9 pre-mixed with the solvent is charged to the reaction vessel followed by addition of the nucleophile, which is charged all at once at the start or added in portions or incrementally during the reaction.

The hydrolysis of esters to acids is a classic chemical manipulation of organic chemistry. In Step 2a the compound of formula 10 can be treated in an aqueous medium with a catalytic amount of a fourth base, such as sodium carbonate, potassium carbonate, sodium hydroxide or potassium hydroxide. The resultant product crystallizes upon cooling to form the compounds of formula 11. Variations of this hydrolysis may be carried out, and although an aqueous alkaline hydrolysis produces satisfactory results, good yields can also be obtained when alkaline aqueous alcohol hydrolysis is used. Alcohols such as t-butyl alcohol, ethyl alcohol, methyl alcohol, isobutyl alcohol, isopropyl alcohol, and the like can be used. The hydrolysis will proceed at temperatures ranging from room temperature to 100° C., preferably at about 25° C. to about 65° C.

The reactants in Step 2a can be added to the reaction vessel (also referred to herein as the reaction “pot”) in concurrently, either together or separately, or they can be added sequentially in either order. The solvent can be added before, during, or after addition of the reactant.

The synthesized compounds of this invention can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art.

The present invention is illustrated by the non-limited examples found below.

### Preparative Example 1

Mesylation of N-Boc-4-piperidinemethanol

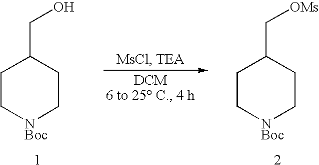

To a 1 L three-necked RBF with overhead stirring was charged with Commercially available Boc protected tert-butyl 4-(hydroxymethyl)piperidine-1-carboxylate (45 g, 209 mmol) 1 and DCM (450 mL), followed by 58.3 mL (418 mmol) of Et₃N (KF 720 ppm). Reaction solution was cooled to 6° C. with an ice/water bath and 17.1 mL (219 mmol) of methanesulfonyl chloride was added via an addition funnel Temperature was controlled bellowed 12° C. (around 20 min). A thin slurry was formed during the addition. After the addition, reaction was slowly warmed to rt. Reaction was monitored by NMR of aliquot. (Both starting material 1 and product 2 can be monitored by HPLC as a single peak (see following HPLC information). However, during the reaction, even if the reaction was completely done based on NMR of aliquot, HPLC always showed a small starting material peak. So it was suggested that both HPLC and NMR should be applied to monitor the reaction.) After around 4 h, reaction was done. (In one run, the reaction was kept at rt for 16 h. The yield of this run is identical as the 4 h reaction. Over time, no significant impurities (such as chloride addition by product) were introduced. Any impurities can be removed by crystallization from heptane.) Reaction solution was filtered through a Solka Floc pad and washed with dichloromethane (Solka Floc filtration was not necessary. TEA HCl salt can be washed away during the aqueous workup). To filtrate was added 200 mL of water (pH around 10) and around 100 mL of 2N HCl slowly to adjust pH to 6-7 (In one run, excess acid was charged incidentally to yield a pH 2-3 and the solution was neutralized right the way to pH 6-7. The Boc group wasn't touched by this incidental event. The same yield was obtained compared to a careful neutralization). Organic layer was separated and washed with water and brine, dried with Na₂SO₄. After concentrated, 200 mL of heptane was charged with rapid stirring. Solid product was crashed out and filtered. Solid was washed with 2×50 mL of heptane to get a pale pink solid. After drying over a N₂/vacuum, the product was formed.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 1: | 1.36 min |
| Times: | Product 2: | 2.16 min |

### Preparative Example 2

### Thioacetate Formation from Mesylate

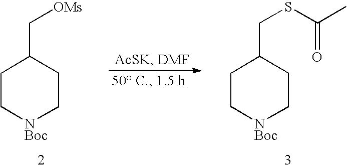

To a 250 mL three-necked RBF with overhead stirring was charged with 9.2 g (31.4 mmol—98 wt % pure) of 2 and 5.12 g (43.9 mmol-98 wt % pure) of AcSK, followed by 92 mL of DMF (KF 160 ppm). Reaction solution was heated to 50° C. under N₂. After 5 min, reaction turned to a thick slurry. Reaction was monitored by HPLC. After 1.5 h, reaction was done (After around 1 h, over 95% conversion was observed. The rest of starting material could be fully converted to product in the next half hour). Reaction was cooled down to 25° C. and was diluted with 120 mL (13 vol) of toluene and 70 mL of water (8 vol). Aqueous layer was separated and LC assay indicated <0.5% mass loss. Organic layer was washed with brine (6 vol) (HPLC indicated no AcSK existed and NMR showed 0.1 equiv of DMF. DMF content was important for the reaction rate. No DMF residue resulted in over 2 days reaction time while 0.5 equiv of DMF caused Pd blacked out and also made reaction complete in 2 days. 0.1 equiv of DMF could be ideal concentration.) to produce compound 3. Organic solution was then concentrated to 50 mL for the next step.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 2: | 2.16 min |
| Times: | Product 3: | 2.92 min |

### Example 1

### Preparation of Compound I as Illustrated by Scheme II

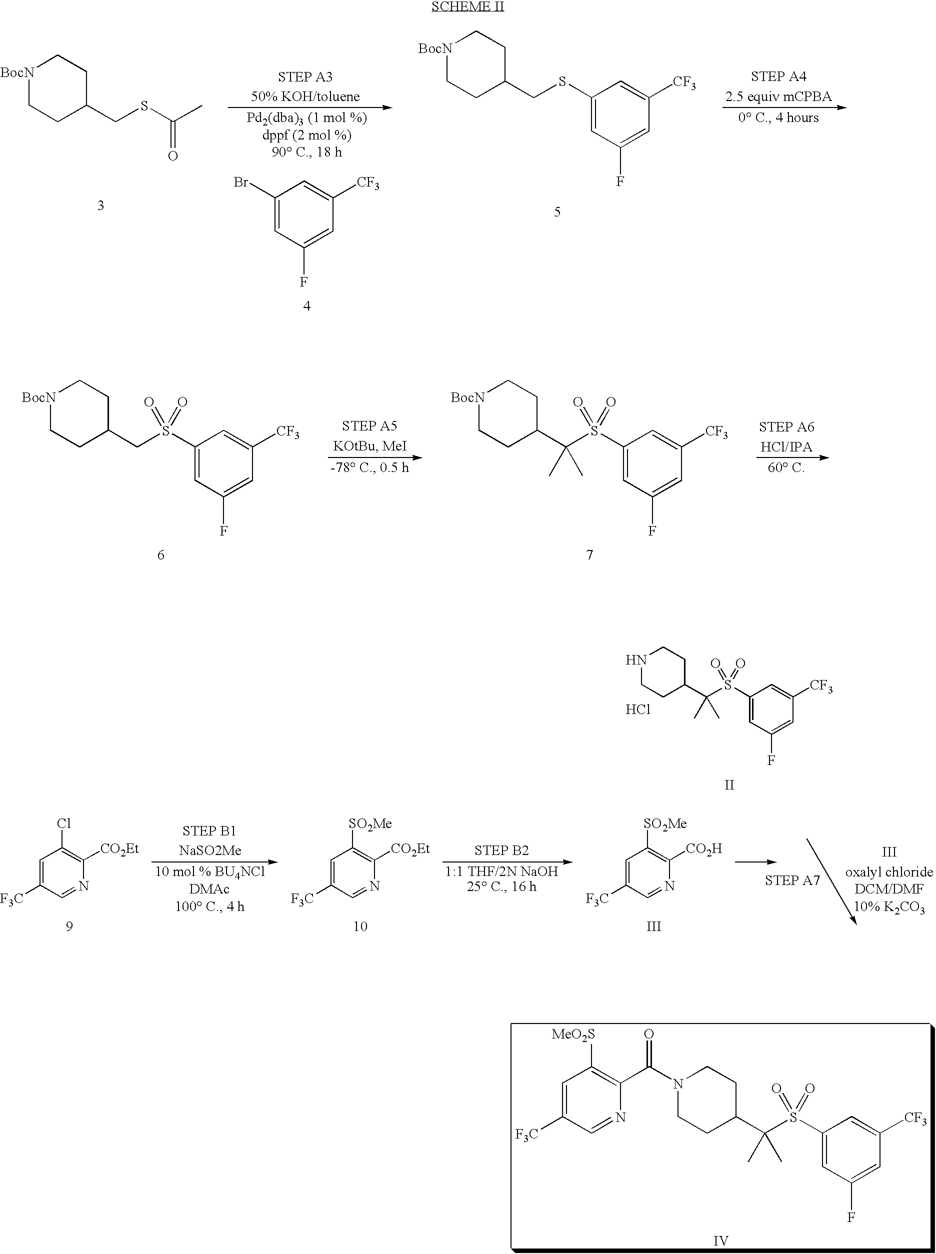

Step A3: Sulfide Formation from Thioacetate

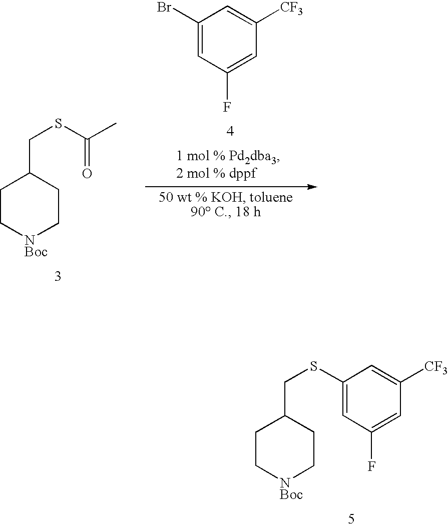

To a 250 mL three-necked RBF with overhead stirring was charged with 8.6 g (31.5 mmol) of 3 in toluene from the last step, toluene (30 mL), 8.6 g (35.2 mmol-98% wt purity) of commercially available aryl bromide, 288 mg (0.31 mmol) of Pd₂ dba₃, 349 mg (0.62 mmol) of dppf, and 50 wt % KOH (13 mL (160 mmol)). Reaction solution was degassed under vacuum/N₂ for 5 min (Vacuum and N₂ was balanced at 600 torr for 5 min). Reaction was then heated to 90° C. for 18 h. Reaction was monitored by HPLC (Reaction conversion in 18 h depended on the work-up solvent from the last step. No thioalcohol (Rf 2.86) was observed during the reaction). Reaction was cooled down to 25° C. and was diluted with 35 mL (4 vol) of toluene and 70 mL of water (8 vol). Organic layer was washed with 2×50 mL of brine (6 vol). LC assay indicated 95% of LCAY and aqueous contained <1% of product Organic solution was treated with 10 mL of EtOAc and 100 wt % of Na₂SO₄. After stirring at rt for 10 min, 50 wt % Ecosorb C-941 and 10 wt % of PL-TMT was charged. Slurry was stirred at 45-50° C. for 1 h. After cooled to room temperature, mixture was filtered through 100 wt % of silica gel to produce compound 5. LC assay indicated 4% mass loss by carbon treatment, and metal analysis indicated a 600 ppm Pd level. Organic solution was then concentrated to 50 mL for the next step.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 3: | 2.92 min |
| Times: | Aryl bromide 4: | 3.18 min |
| | Product 5: | 3.90 min |
| | Thioalcohol intermediate: | 2.86 min |

Step A4: Oxidation of Thioether to Sulfone Using mCPBA

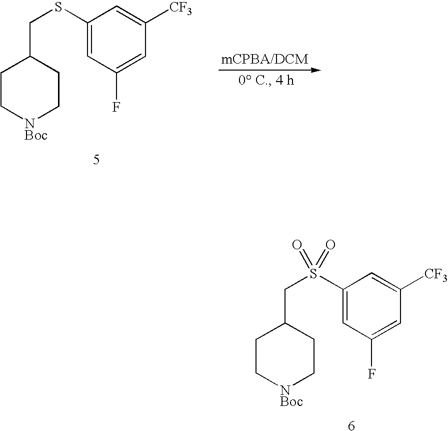

To 250 mL 3-necked RBF was charged with the toluene solution of 5 (9.3 g (23.6 mmol) of 5) followed by 60 mL of DCM. The solution was cooled to −10° C. m-CPBA (12.7 g, 56.7 mmol-77 wt % purity) was added portionwise while maitaining the internal temperature below −4° C. The reaction is exothermic upon addition of mCPBA until ˜50% of the mCPBA is added. Temperature was −6° C. after mCPBA was all added. At this point, the color of the reaction changed from orange to yellow slurry. Sulfide 5 was all consumed at this point and sulfoxide was the main product by HPLC. The slurry was warmed to 4° C. in an ice bath. After aging at 4-5° C. for 70 minutes, the ratio of 6/sulfoxide was 92/8. After 3 hours, 6/sulfoxide=97/3. The reaction seems to stall at 97% conversion and 0.15 equiv mCPBA was added and the reaction aged at 10° C. for 40 minutes to achieve >99:1 6:sulfoxide (10.0 g (23.6 mmol)).

The reaction was quenched by the addition of 45 mL (5 vols) of 10 wt % Na₂S₂O₃. The mixture was then diluted with water (45 mL, 5 vols) and EtOAc (145 mL, 15 vols). The layers were separated and washed with 10 wt % K₂CO₃ (2×5 vols) and brine (1×5 vols). The assay yield of 6 was determined to be >95% by HPLC analysis.

The yellow solution was concentrated under vacuum at 50-60° C. to around 4 volumes of solvent per mass of reagent and charged with 35 mL of heptane. Solution was then concentrated to 3-4 vol. The sulfone crystallized during the solvent switch. After solvent switch, solution was heated to 75° C. to dissolve all solid and cooled slowly with agitation to room temperature (rt). At around 50° C., crystallization started occurring. After cooled to rt, slurry was cooled in an ice bath to 3° C. before filtration. Mother liquor contained ˜6 mol % EtOAc, 27 mol % toluene relative to heptane. Solid was washed with 30 mL of cold heptane (0 C) and dried in N2/vacuum at 40° C. for 2 hours (h) to give an off white solid,

Mother liquor contained around 4% of product (LCA) with most of impurities.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 5: | 3.90 min |
| Times: | Sulfone product 6: | 3.11 min |
| | Sulfoxide intermediate: | 2.92 min |

### Step A5: Dimethylation of Sulfone

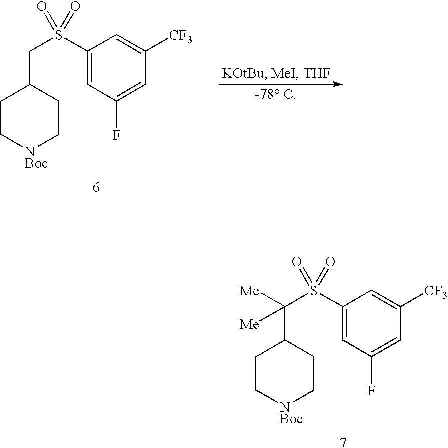

To a 100 mL round bottom flask (RBF) with a stir bar was charged with 2.0 g (4.5 mmol) of 6 followed by 12 mL of THF (50 ppm water). The resulting solution was cooled to −78° C. with a dry ice/acetone bath and charged with 13.6 mL (3.0 equiv) of 1 M KOtBu in THF. The temperature was controlled below −60° C. (If the reaction temperature is too high (>40-50° C.), an over-addition by-product will be observed.). After 1 min, to the resulting solution was added 0.59 mL (9.5 mmol) of Met (2.1 equiv) slowly to control temperature below −60° C. HPLC indicated complete consumption of starting material sulfone, but with 20-30% of mono-methylated intermediate. At the same temperature, to the reaction was charged with 4.5 mL (1 equiv) of KOtBu (temperature below −60° C.) HPLC indicated <3% of mono-methylated intermediate. At the same temperature, 0.9 mL (0.2 equiv) of KOtBu was charged (temperature below −60° C.). After 1 min, HPLC indicated <1% of mono-methylated intermediate left. The reaction produced 2.05 g (4.5 mmol) of compound 7). Reaction was then warmed to 0 C before quenching with 15 mL of 10% NH₄Cl and 20 mL of EtOAc. After the phase cut, organic solution was washed with 15 mL of brine, and then was solvent switched to IPA (20 mL) for the next step.

Over-addition by-product:

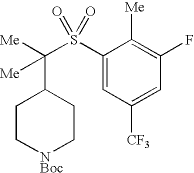

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 6: | 3.11 min |
| Times: | Product 7: | 3.35 min |
| | Mono-methyl intermediate: | 3.24 min |
| | Over-addition by product: | 3.59 min |

Step A6: Deprotection of Boc Group with HCl in IPA

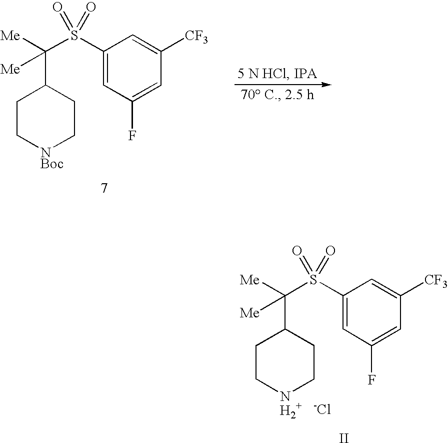

To a 100 mL RBF with a stir bar was charged with 2.17 g (4.79 mmol) 7 in 22 mL of IPA. To the resulting solution was charged with 5 N HCl in IPA (2.9 mL (14.4 mmol)). The reaction was then heated to 70° C. for 2 h. HPLC indicated complete conversion and product was formed as a white solid. After reaction was cooled to 5° C. using an ice bath, product was filtered and washed with 20 mL IPA followed by 15 mL of MTBE. After dried over N2/vacuum at 40° C. for 2.5 h, reaction gave compound II as a white solid (95% corrected yield over two steps). LC indicated 98% LCAP and NMR gave a 95 wt %

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 7: | 3.35 min |
| Times: | Product II: | 1.22 min |

Step B1: C—S Formation of ethyl 3-chloro-5-trifluoromethylpyridine-2-carboxylate.

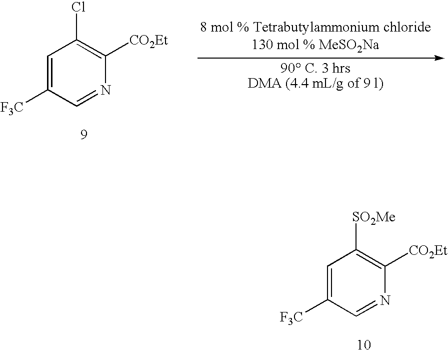

A 100 mL RBF was charged commercially available 9 (6.80 g, 26.8 mmol), tetrabutylammonium chloride (0.60 g, 2.159 mmol) and methanesulfinic acid sodium salt (4.2 g, 35.0 mmol), followed by N,N-dimethylacetamide (30.0 ml). The reaction was mechanically stirred in a 90° C. oil bath for 2 hr. TLC showed a slight starting material spot and HPLC gave a product/starting material ratio of 7.2. 3 hr TLC showed a complete reaction. The reaction was cooled down to rt to give a cloudy mixture. 60 mL water was added slowly while mechanically stirred, to give acolorless thick slurry. The slurry was then filtered and washed with water (80 mL, in three portions), dried at 60° C. under vacuum to afford 10 as a colorless fluffy solid.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 9: | 2.90 min |
| Times: | Product 10: | 2.48 min |

Step B2: Hydrolysis of ethyl 3-methanesulfonyl-5-trifluoromethylpyridine-2-carboxylate.

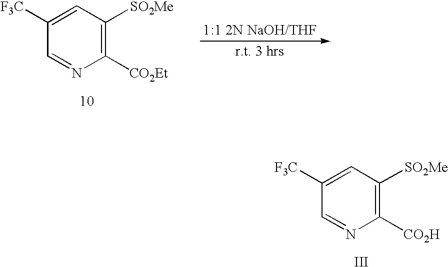

To slightly tan fluffy solid 10 (14.60 g, 49.1 mmol) in a 500 mL three-neck RBF was added 50 mL THF and 50 mL 2N NaOH. The mixture was mechanically stirred at rt for 3 hours (hr). TLC showed a complete reaction. 8.5 mL conc. HCl was added to turn to pH˜2 and followed by 50 mL EtOAc. The mixture was stirred and separated. HPLC analysis showed an aq. (˜60 mL) loss of less than 1 wt %. The organic layer was transferred into a 500 mL three-neck RBF, mechanically stirred in a 50° C. oil bath and distilled under house vacuum with the addition of heptane (110 mL), resulting in a thick slurry. NMR showed the mother liquor was 1:2:6 THF/EtOAc/heptane mixture. The slurry was filtered and washed with heptane (×3). The filtered cake was dried at 60° C. under house vacuum overnight to afford III as a slightly tinted fluffy colorless solid. NMR with internal standard showed a purity of 100 wt %.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Starting material 10: | 2.48 min |
| Times: | Product 11: | 1.22 min |

### Step A7: Amide Bond Formation

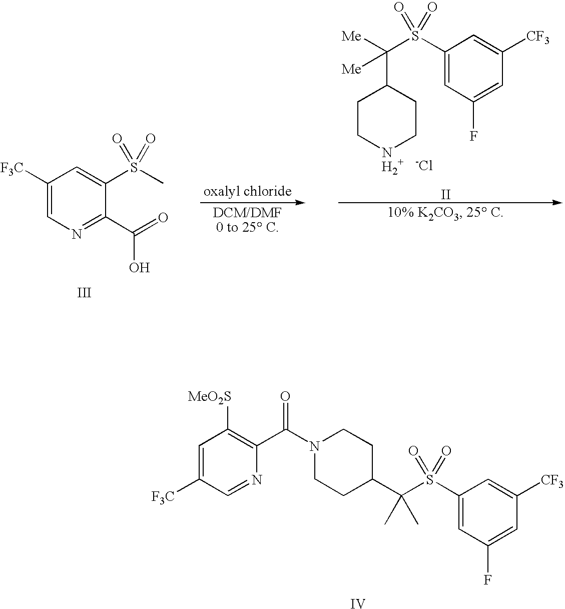

To a 100 mL RBF with a stir bar was charged with acid III (1.1 g, 4.02 mmol->99 wt % purity) in 22 mL of DCM. To the resulting solution at 3° C. was charged with oxalyl chloride (0.384 mL, 4.4 mmol, >98% purity). The reaction was then slowly warmed up to rt. During this process, reaction occurred slowly with the gas release. After the temperature reached to 20° C., HPLC indicated fully conversion to acyl chloride and reaction turned from a slurry to clean light yellow color solution. To the resulting solution at rt was added amine II (1.5 g, 3.6 mmol-95 wt % purity) in one portion. Temperature increased by 2° C. To the resulting solution was charged 10% K₂CO₃ (8 mL) dropwise. Reaction occurred right the way with gas releasing. After addition, HPLC indicated complete conversion. To the reaction, was charged 15 mL of K₂CO₃. After the phase cut, organic layer was washed by 15 mL of K₂CO₃ and 10 mL of brine. Solution was then transferred to a 100 mL 3-necked and solvent switch to IPA.

After product solution was solvent switched to 15 mL IPA at 20° C., some sticky solid was crashed out. After heating at 60° C., sticky solid almost dissolved and then transferred to white crystalline solid. This solid didn't dissolve even at 70° C. After cooled to 2° C. using ice bath, solid was filtered and washed with 12 mL of 2° C. IPA and 15 mL of hexane and dried over N2/vacuum at 40° C. for 2 h gave compound IV as a white solid.

### HPLC Method

| Column: | Agilent Zorbax | | |
|---|---|---|---|
| | (Eclipse Rapid Resolution HT) | | |
| | 4.6 mm × 50 mm (1.8 micron) | | |
| Temperature: | 40° C. | | |
| Mobile Phase: | A: 0.1% aq. H₃PO₄, B: MeCN | | |
| Flowrate: | 1.5 mL/min | | |
| Max Pressure: | 400 bar | | |
| Detection: | UV Absorbance @ 205 nm | | |
| Injection Volume: | 5 μL | | |
| Run time: | 6 min | | |
| Post time: | 1 min | | |
| | Time | % A | % B |
| Gradient: | 0 | 70 | 30 |
| | 3 | 5 | 95 |
| | 6 | 5 | 95 |

| | | |
|---|---|---|
| Typical Retention | Amine salt II: | 1.22 min |
| Times: | Acid III: | 0.57 min |
| | Methyl ester | 1.82 min |
| | (methanol quenching) | |
| | Compound I: | 2.97 min |

While certain preferred embodiments of the invention have been described herein in detail, numerous alternative embodiments are contemplated as falling within the scope of the appended claims. Consequently the invention is not to be limited thereby.

The abbreviations used herein have the following meanings (abbreviations not shown here have their meanings as commonly used unless specifically stated otherwise): Ac (acetyl), Bn (benzyl), Boc (tertiary-butoxy carbonyl), Bop reagent (benzotriazol-1-yloxy)tris(dimethylamino)phosonium hexafluorophosphate, DMF (N,N-dimethylformamide), DPPF (1,1′-bisdiphenylphosphino ferrocene), EDC (1-(3-dimethylaminopropyl)-3-ethylcarbodiimide hydrochloride), Et₃N (triethylamine), HOBt (1-hydroxybenzotriazole), LAH (lithium aluminum hydride), Ms (methanesulfonyl; mesyl; or SO₂Me), MsO (methanesulfonate or mesylate), mCPBA (meta-chloro perbenzoic acid), Ph (Phenyl), r.t. or rt (room temperature), Rac (Racemic), TFA (trifluoroacetic acid), THF (Tetrahydrofuran), TLC (thin layer chromatography), wt % (weight percent), HCl (hydrochloride), RBF (round bottom flask), DCM (dichloromethane), mL (milliliter), ppm (parts per million), NMR (nuclear magnetic resonance), uv-(ultraviolet), vol (volume), min (minute), rt (room temperature), IPA (isopropanol), g (gram(s)), eq=equiv (equivalent), Me (methyl), Et (ethyl), n-Pr (normal propyl), i-Pr (isopropyl), n-Bu (normal butyl), i-Butyl (isobutyl), s-Bu (secondary butyl), t-Bu (tertiary butyl), c-Pr (cyclopropyl), c-Bu (cyclobutyl), c-Pen (cyclopentyl), c-Hex (cyclohexyl), and AcSK=KSAc=

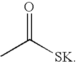

Unless specifically stated otherwise, the experimental procedures were performed under the following conditions: All operations were carried out at room or ambient temperature; that is, at a temperature in the range of 18-25° C. Inert gas protection was used when reagents or intermediates were air and moisture sensitive. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) or by high-pressure liquid chromatography-mass spectrometry (HPLC-MS), and reaction times are given for illustration only. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. In addition, “Ar” signifies an aromatic signal. Chemical symbols have their usual meanings.

What is claimed is:

1. A compound which is 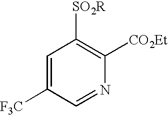
or pharmaceutically acceptable salt thereof, wherein R is selected from the group consisting of R is C₁₋₆alkyl, (CH₂)ₙC₁₋₄-fluoroalkyl, C₃₋₇cycloalkyl, and NR¹⁰R¹¹.

2. The compound according to claim 1 which is:

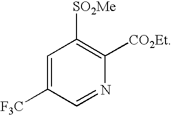

3. A compound which is: 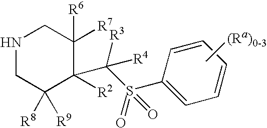
or a pharmaceutically acceptable salt thereof wherein:
R² is H, C₁₋₄ alkyl and C₁₋₄-perfluoroalkyl, C₃₋₅-cycloalkyl, C₆₋₁₀ aryl, C₅₋₁₀ heteroaryl, F, Cl, CN, NR¹⁰R¹¹, wherein said alkyl, cycloalkyl, aryl and heteroaryl is optionally substituted with one to three groups of R^{a};
R³ and R⁴ are each and independently selected from H, or C₁₋₆ alkyl, C₁₋₄-perfluoroalkyl, C₃₋₇-cycloakyl, C₆₋₁₀ aryl, C₅₋₁₀ heteroaryl, F, Cl, CN, OR¹⁰, NR¹⁰R¹¹, SO₂R¹⁰SO₂NR¹⁰R¹¹, CO₂R¹⁰, CONHR¹⁰, CONR¹⁰R¹¹, or R³ and R⁴ join to form a 3-7 member carbocyclic or heterocyclic ring, wherein said alkyl, cycloalkyl, heterocycle, aryl and heteroaryl is optionally substituted with one to three groups of R^{a};
R⁶, R⁷, R⁸, and R⁹ independently represent H, C₁₋₄alkyl and C₁₋₄ perfluoroalkyl, C₃₋₆-cycloalkyl, C₆₋₁₀ aryl, C₅₋₁₀ heteroaryl, F, Cl, CN, OR¹⁰, NR¹⁰R¹¹, or R⁸ and R⁹ combined with the carbon atom they are attached to can form C(O);
R¹⁰ and R¹¹ are each and independently selected from H, or C₁₋₆alkyl, (CH₂)ₙC₁₋₄-fluoroalkyl, C₃₋₇cycloalkyl, C₆₋₁₀ aryl, C₅₋₁₀ heteroaryl, or R¹⁰ and R¹¹ join to form a 3-7 member carbocyclic or heterocyclic ring with the atom to which they are attached; said alkyl, aryl, or heteroaryl optionally substituted with 1 to 3 groups of R^{a},
n represents 0 to 6, and
R^{a} represents C₁₋₆ alkyl, C₃₋₇ cycloalkyl, C₁₋₄-fluoroalkyl, C₆₋₁₀ aryl, C₅₋₁₀ heteroaryl, halogen, CN, —OCF₃, —OCHF₂, —C(O)CF₃, —C(OR¹⁰)(CF₃)₂, SR¹⁰, —OR¹⁰, NR¹⁰R¹¹, SOR¹⁰, SO₂R¹⁰, NR¹⁰COR¹¹, NR¹⁰COOR¹¹, NR¹⁰CONR¹⁰R¹¹, NR¹⁰SO₂NR¹⁰R¹¹, SO₂NR¹⁰R¹¹, NR¹⁰SO₂R¹¹, CO₂R¹⁰, CONR¹⁰R¹¹, said aryl and heteroaryl optionally substituted with 1 to 3 groups of C₁₋₆ alkyl, C₃₋₇ cycloalkyl, halogen, CF₃, CN or OR¹⁰;

4. The compound according to claim 3 represented by: 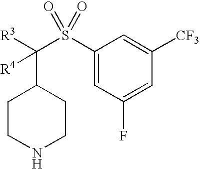
or pharmaceutically acceptable salt thereof, wherein:
R³ and R⁴ are independently C₁₋₆ alkyl, C₁₋₄-perfluoroalkyl, C₃₋₇ cycloalkyl C₆₋₁₀ aryl, C₅₋₁₀heteroaryl, F, Cl, CN, OR¹⁰, NR¹⁰R¹¹, SO₂R¹⁰, SO₂NR¹⁰R¹¹, CO₂R¹⁰, CONHR¹⁰, CONR¹⁰R¹¹, or R³ and R⁴ join to form a 3-7 member carbocyclic or heterocyclic ring, wherein said alkyl, cycloalkyl, heterocycle, aryl and heteroaryl is optionally substituted with one to three groups of R^{a};
R¹⁰ and R¹¹ are each and independently selected from H, or C₁₋₆alkyl, (CH₂)ₙC₁₋₄-fluoroalkyl, C₃₋₇cycloalkyl, C₆₋₁₀ aryl, C₅₋₁₀ heteroaryl, or R¹⁰ and R¹¹ join to form a 3-7 member carbocyclic or heterocyclic ring with the atom to which they are attached; said alkyl, aryl, or heteroaryl optionally substituted with 1 to 3 groups of R^{a},
n represents 0 to 6, and
R^{a} represents C₁₋₆ alkyl, C₃₋₇ cycloalkyl, C₁₋₄-fluoroalkyl, C₆₋₁₀ aryl, C₅₋₁₀ heteroaryl, halogen, CN, —OCF₃, —OCHF₂, —C(O)CF₃, —C(OR¹⁰)(CF₃)₂, SR¹⁰, —OR¹⁰, NR¹⁰R¹¹, SOR¹⁰, SO₂R¹⁰, NR¹⁰COR¹¹, NR¹⁰COOR¹¹, NR¹⁰CONR¹⁰R¹¹, NR¹⁰SO₂NR¹⁰R¹¹, SO₂NR¹⁰R¹¹, NR¹⁰SO₂R¹¹, CO₂R¹⁰, CONR¹⁰R¹¹, said aryl and heteroaryl optionally substituted with 1 to 3 groups of C₁₋₆ alkyl, C₃₋₇ cycloalkyl, halogen, CF₃, CN or OR¹⁰.

5. The compound according to claim 4 wherein R³ and R⁴ independently are C₁₋₆ alkyl or a pharmaceutically acceptable salt thereof.

6. The compound according to claim 5 which is: 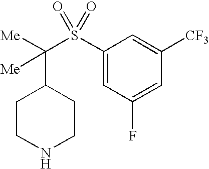
or a pharmaceutically acceptable salt thereof.
